(19) United States
(12) Patent Application Publication
(10) Pub. No.: US 2022/0403431 A1
(43) Pub. Date: Dec. 22, 2022

(54) GLYCOMINIMIZED BACTERIAL HOST CELLS

(71) Applicant: INBIOSE N.V., Zwijaarde (BE)

(72) Inventors: Joeri Beauprez, Zwijaarde (BE); Sofia De Maeseneire, Zwijaarde (BE); Nico Snoeck, Zwijaarde (BE)

(21) Appl. No.: 17/904,213

(22) PCT Filed: Feb. 12, 2021
(86) PCT No.: PCT/EP2021/053500

§ 371 Date: Aug. 12, 2022

(30) Foreign Application Priority Data
Feb. 14, 2020 (BE) 20205097

(51) Int. Cl. C12P 19/04; C12P 19/02; C12N 1/20; C12N 15/90

(52) U.S. Cl. CPC C12P 19/04; C12P 19/02; C12N 1/20; C12N 15/90

(57) ABSTRACT

This disclosure is in the technical field of synthetic biology and metabolic engineering. The disclosure provides engineered viable bacteria having a reduced or abolished synthesis of poly-N-acetyl-glucosamine (PNAG), Enterobacterial Common Antigen (ECA), cellulose, colanic acid, core oligosaccharides, Osmoregulated Periplasmic Glucans and Glucosylglycerol (O), glycan, and trebalose. The disclosure further provides methods for the production of bioproduct by the viable bacteria and uses thereof. Furthermore, the disclosure is in the technical field of fermentation of metabolically engineered microorganisms producing bioproduct.

### CROSS-REFERENCE TO RELATED APPLICATIONS

This application is a national phase entry under 35 U.S.C. § 371 of International Patent Application PCT/EP2021/053500, filed Feb. 12, 2021, designating the United States of America and published as International Patent Publication WO 2021/160830 A1 on Aug. 19, 2021, which claims the benefit under Article 8 of the Patent Cooperation Treaty to Belgian Patent Application Serial No. 2020/5097, filed Feb. 14, 2020.

### TECHNICAL FIELD

The disclosure is in the technical field of synthetic biology and metabolic engineering. The disclosure provides engineered viable bacteria having a reduced or abolished synthesis of poly-N-acetyl-glucosamine (PNAG), Enterobacterial Common Antigen (ECA), cellulose, colanic acid, core oligosaccharides, Osmoregulated Periplasmic Glucans and Glucosylglycerol (OPG), glycan, and trehalose. The disclosure further provides methods for the production of bioproduct by the viable bacteria and uses thereof. Furthermore, the disclosure is in the technical field of fermentation of metabolically engineered microorganisms producing bioproduct.

### BACKGROUND

The cell wall of bacteria is an essential structure that provides the cell support and protects the cell from mechanical stress or damage from osmotic rupture or lysis. The cell wall further provides bacteria important ligands for adherence and receptor sites for viruses or antibiotics. The cell wall of Gram-negative bacteria is composed of a single layer of peptidoglycan surrounded by the outer membrane that contains lipo- and exopolysaccharide molecules in addition to proteins and phospholipids. Glycosyltransferases are a huge enzyme family that are involved in the synthesis of the extracellular polysaccharide matrix including poly-N-acetylglucosamine (PNAG), colanic acid, the enterobacterial common antigen (ECA) and in the O-antigen and core oligosaccharides of the lipopolysaccharide outer membrane. Glycosyltransferases catalyze the transfer of a sugar moiety from an activated sugar donor onto saccharide or non-saccharide acceptors (Coutinho et al., J. Mol. Biol. 328 (2003), 307-317). These glycosyltransferases are also the source for biotechnologists to synthesize bioproducts, e.g., specialty saccharides (such as disaccharides, oligosaccharide and polysaccharides), glycolipids and glycoproteins as described e.g., in WO2013/087884, WO2012/007481, WO2016/075243 or WO2018/122225. These glycosyltransferases may thus interfere with the intended product, intermediates or the used substrate causing unwanted side-reactions and may eventually create a product loss. Altering the host's glycosyltransferases to improve bioproduct production may lead to a severely altered cell wall of the host and/or slimy cell phenotypes, reduced cell fitness, altered osmotic and/or antibiotic sensitivity of the production host, resulting in inefficient and expensive fermentation processes and/or a difficult and costly down-stream processing to obtain the desired product.

### SUMMARY OF DISCLOSURE

Provided herein are tools and methods by means of which bioproducts can be produced in an efficient, time and cost-effective way and which yield high amounts of the desired product with minimized interference of the host's natural metabolism.

Also provided herein are a cell and a method for the production of a bioproduct wherein the cell is genetically modified for the production of the products and additionally lack non-essential glycosyltransferases that are involved in the synthesis of the extracellular polysaccharide matrix and lipopolysaccharide outer membrane.

Surprisingly it has now been found that the genetically modified microorganisms with reduced or abolished synthesis of poly-N-acetyl-glucosamine (PNAG), Enterobacterial Common Antigen (ECA), cellulose, colanic acid, core oligosaccharides, Osmoregulated Periplasmic Glucans and Glucosylglycerol (OPG), glycan, and trehalose provide for newly identified glycominimized microorganisms having a similar or positive effect on fermentative production of a bioproduct, in terms of yield, productivity, specific productivity and/or growth speed. Expression of heterologous genes to the glycominimized microorganisms for synthesis of bioproducts did not affect the performance of the glycominimized cell. In the production of bioproducts e.g., glycosylated products like oligosaccharides, little to no effect was observed on the strain's fitness, as exemplified with the growth rate, nor on the strain's osmotic and antibiotic sensitivity, even when compared to a non-glycominimized reference cell.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the disclosure disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

According to the disclosure, the term “glycominimized (bacterial host) cell” refers to a cell that has reduced or abolished synthesis of non-essential glycosyltransferases involved in the synthesis of poly-N-acetyl-glucosamine (PNAG), Enterobacterial Common Antigen (ECA), cellulose, colanic acid, core oligosaccharides, Osmoregulated Periplasmic Glucans (OPGs) and Glucosylglycerol, glycan, and trehalose and that preferably has reduced or abolished synthesis of further predicted non-essential glycosyltransferases.

According to the disclosure, the term “glycosyltransferase” refers to a protein that catalyzes the transfer of a carbohydrate acceptor from an activated sugar nucleotide donor enabling extension and branching of glycans and glycoconjugates to form di-, oligo-, polysaccharides, lipo(poly)saccharides or peptidoglycan (Mestrom et al., Int. J. Mol. Sci. 20 (2019), 5263).

The term “glycosyltransferase encoding gene(s)” as used herein encompasses polynucleotides that include a sequence encoding a glycosyltransferase of the disclosure. The term also encompasses polynucleotides that include a single continuous region or discontinuous regions encoding the glycosyltransferase (for example, interrupted by integrated phage or an insertion sequencing or editing) together with additional regions that also may contain coding and/or non-coding sequences.

According to the disclosure, the term “non-essential and predicted non-essential glycosyltransferase” refers to a glycosyltransferase that is not critical for the host cell for its survival in rich growth media. The term “non-essential and predicted non-essential glycosyltransferase genes” refer to genes encoding for glycosyltransferases and polypeptides predicted to be glycosyltransferases that do not lead to a lethal phenotype when inactivated from the host genome.

The term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is a mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the disclosure. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic make-up and is replicated. “Mutant” cell or microorganism as used within the context of the disclosure refers to a cell or microorganism, which is genetically engineered or has an altered genetic make-up.

The term “endogenous,” within the context of the disclosure refers to any polynucleotide, polypeptide or protein sequence, which is a natural part of a cell and is occurring at its natural location in the cell chromosome. The term “exogenous” refers to any polynucleotide, polypeptide or protein sequence, which originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “polynucleotide encoding a polypeptide” as used herein encompasses polynucleotides that include a sequence encoding a polypeptide of the disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “modified expression” of a gene relates to a change in expression compared to the wild type expression of the gene in any phase of biosynthesis of a product. The modified expression is either a lower or higher expression compared to the wild type, wherein the term “higher expression” is also defined as “overexpression” of the gene in the case of an endogenous gene or “expression” in the case of a heterologous gene that is not present in the wild type strain. Lower expression is obtained by means of common well-known technologies for a skilled person (such as but not limited to the usage of siRNA, CRISPR, CRISPRi, riboswitches, recombineering, homologous recombination, ssDNA mutagenesis, RNAi, miRNA, asRNA, mutating genes, knocking-out genes, transposon mutagenesis, . . . ) which are used to change the genes in such a way that they are less-able (i.e., statistically significantly ‘less-able’ compared to a functional wild-type gene) or completely unable (such as knocked-out genes) to produce functional final products. Overexpression or expression is obtained by means of common well-known technologies for a skilled person, wherein the gene is part of an “expression cassette,” which relates to any sequence in which a promoter sequence, untranslated region sequence (UTR, containing either a ribosome binding sequence or Kozak sequence), a coding sequence and optionally a transcription terminator is present, and leading to the expression of a functional active protein. The expression is either constitutive or conditional or regulated.

The term “constitutive expression” is defined as expression that is not regulated by transcription factors other than the subunits of RNA polymerase (e.g., the bacterial sigma factors) under certain growth conditions. Non-limiting examples of such transcription factors are CRP, LacI, ArcA, Cra, IclR in E. coli. These transcription factors bind on a specific sequence and may block or enhance expression in certain growth conditions. RNA polymerase binds a specific sequence to initiate transcription, for instance, via a sigma factor in prokaryotic hosts.

The term “regulated expression” is defined as expression that is regulated by transcription factors other than the subunits of RNA polymerase (e.g., bacterial sigma factors) under certain growth conditions. Examples of such transcription factors are described above. Commonly expression regulation is obtained by means of an inducer, such as but not limited to IPTG, arabinose, rhamnose, fucose, allo-lactose or pH shifts or temperature shifts or carbon depletion or substrates or the produced product. Regulated expression can also be obtained by using riboswitches. A riboswitch is defined to be part of the messenger RNA that folds into intricate structures that block expression by interfering with translation. Binding of an effector molecule induces conformational change(s) permitting regulated expression post-transcriptionally.

The term “wild type” refers to the commonly known genetic or phenotypical situation as it occurs in nature.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The terms “cell genetically modified for the production of glycosylated product” within the context of the disclosure refers to a cell of a microorganism, which is genetically manipulated to comprise at least one of i) a gene encoding a glycosyltransferase necessary for the synthesis of the glycosylated, ii) a biosynthetic pathway to produce a nucleotide donor suitable to be transferred by the glycosyltransferase to a carbohydrate precursor, and/or iii) a biosynthetic pathway to produce a precursor or a mechanism of internalization of a precursor from the culture medium into the cell where it is glycosylated to produce the glycosylated product.

The terms “nucleic acid sequence coding for an enzyme for glycosylated product synthesis” relates to nucleic acid sequences coding for enzymes necessary in the synthesis pathway to the glycosylated product.

The term ‘fucosylated LNT III” within the context of the disclosure refers to fucosylated Lacto-N-neo-tetraose (LNnT) or fucosyllacto-N-neotetraose III or Gal(b1-4)[Fuc(a1-3)]GlcNAc(b1-3)Gal(b1-4)Glc, also known as lacto-N-fucopentaose III or FLNP III or Le^{x}-lactose or Lewis-X pentasaccharide.

The term bioproduct as used herein is any product that can be synthesized in a biological manner, i.e., via enzymatic conversion, microbial biosynthesis, cellular biosynthesis.

Examples of bioproducts are:

1) Small organic molecules, such as but not limited to organic acids, alcohols, amino acids; proteins, such as but not limited to enzymes, antibodies, single cell protein, nutritional proteins, albumins, lactoferrin, glycolipids and glycopeptides; antibiotics, such as but not limited to antimicrobial peptides, polyketides, penicillins, cephalosporins, polymyxins, rifampycins, lipiarmycins, quinolones, sulfonamides, macrolides, lincosamides, tetracyclines, aminoglycosides cyclic lipopeptides (such as daptomycin), glycylcyclines (such as tigecycline), oxazolidinones (such as linezolid), lipiarmycins, fidaxomicin; lipids, such as but not limited to arachidonic acid, docosahexaenic acid, linoleic acid, Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA), Clupanodonic acid, Tetracosapentaenoic acid, Tetracosahexaenoic acid (Nisinic acid), Flavonoids, glycolipids, ceramides, sphingolipids, carbohydrates, monosaccharides, phosphorylated monosaccharides, activated monosaccharides, disaccharides, polysaccharides, oligosaccharides such as but not limited to human milk oligosaccharides, glycosaminoglycans, chitosans, chondrotoines, heparosans, Glucuronylated oligosaccharides;

2) A mammalian milk oligosaccharide as defined herein;

3) A ‘sialylated oligosaccharide’ as defined herein;

4) A ‘fucosylated oligosaccharide’ as defined herein;

5) A ‘neutral oligosaccharide’ as defined herein;

6) A monosaccharide as defined herein;

7) A disaccharide or oligosaccharide containing any one or more monosaccharide as described herein.

The term polyol as used herein is an alcohol containing multiple hydroxyl groups, for example, glycerol, sorbitol, or mannitol. The term “sialic acid” as used herein refers to the group comprising sialic acid, neuraminic acid, N-acetylneuraminic acid and N-Glycolylneuraminic acid.

The term “glycosylated product” as used herein refer to the group of molecules comprising at least one monosaccharide as defined herein. More, in particular, the bioproduct is chosen from the list comprising, preferably consisting of, monosaccharide, phosphorylated monosaccharide, activated monosaccharide, disaccharide, oligosaccharide, glycoprotein and glycolipid.

The term “monosaccharide” as used herein refers to saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O-[(R)-1-carboxyethyl]-2-deoxy-D-glucopyranose, 2-Acetamido-3-O-[(R)-carboxyethyl]-2-deoxy-D-glucopyranose, 2-Glycolylamido-3-O-[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, glucose, galactose, N-acetylglucosamine, glucosamine, mannose, xylose, N-acetylmannosamine, N-acetylneuraminic acid, N-glycolylneuraminic acid, a sialic acid, N-acetylgalactosamine, galactosamine, fucose, rhamnose, glucuronic acid, gluconic acid, fructose and polyols.

The term “phosphorylated monosaccharide” as used herein refers to one of the above listed monosaccharides, which is phosphorylated. Examples of phosphorylated monosaccharides include but are not limited to glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The term “activated monosaccharide” as used herein refers to activated forms of monosaccharides, such as the monosaccharides as listed here above. Examples of activated monosaccharides include but are not limited to GDP-fucose, GDP-mannose, CMP-N-acetylneuraminic acid, CMP-N-glycolylneuraminic acid, UDP-glucuronate, UDP-N-acetylgalactosamine, UDP-glucose, UDP-galactose, CMP-sialic acid and UDP-N-acetylglucosamine. Activated monosaccharides, also known as nucleotide sugars, act as glycosyl donors in glycosylation reactions. Those reactions are catalyzed by a group of enzymes called glycosyltransferases.

The term “disaccharide” as used herein refers to a saccharide polymer containing two simple sugars, i.e., monosaccharides. Such disaccharides contain monosaccharides as described above and are preferably selected from the list of monosaccharides as used herein above. Examples of disaccharides comprise lactose, N-acetyllactosamine, and Lacto-N-biose.

“Oligosaccharide” as the term is used herein and as generally understood in the state of the art, refers to a saccharide polymer containing a small number, typically three to fifteen, of simple sugars, i.e., monosaccharides. Preferably the oligosaccharide as described herein contains monosaccharides selected from the list as used herein above. Examples of oligosaccharides include but are not limited to neutral oligosaccharides, fucosylated oligosaccharides, sialylated oligosaccharide, Lewis-type antigen oligosaccharides, mammalian milk oligosaccharides and human milk oligosaccharides.

As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated milk oligosaccharides, neutral milk oligosaccharide and/or sialylated milk oligosaccharides.

As used herein the term “Lewis-type antigens” comprise the following oligosaccharides: H1 antigen, which is Fucα1-2Galβ1-3GlcNAc, or in short 2′FLNB; Lewis^{a}, which is the trisaccharide Galβ1-3[Fucα1-4]GlcNAc, or in short 4-FLNB; Lewis^{b}, which is the tetrasaccharide Fucα1-2Galβ1-3[Fucα1-4]GlcNAc, or in short DiF-LNB; sialyl Lewis^{a} which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′fucosyl-N-acetyl-lactosamine, in short 2′FLacNAc; Lewis^{x}, which is the trisaccharide Galβ1-4[Fucα1-3]GlcNAc, or otherwise known as 3-Fucosyl-N-acetyl-lactosamine, in short 3-FLacNAc, Lewis^{y}, which is the tetrasaccharide Fucα1-2Galβ1-4[Fucα1-3]GlcNAc and sialyl Lewis^{x} which is

5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

As used herein, a ‘sialylated oligosaccharide’ is to be understood as a charged sialic acid containing oligosaccharide, i.e., an oligosaccharide having a sialic acid residue. It has an acidic nature. Some examples are 3-SL (3′-sialyllactose), 3′-sialyllactosamine, 6-SL (6′-sialyllactose), 6′-sialyllactosamine, oligosaccharides comprising 6′-sialyllactose, SGG hexasaccharide (Neu5Acα-2,3Galβ-1,3GalNacβ-1,3Gala-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1a Neu5Acα-2,3Galβ-1,3GalNAcβ-1,4(Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc β-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAc β-1,4(Neu5Acα-2,8Neu5Acα-2,8Neu5Acα2,3)Galβ-1,4Glc, GP1c Neu5Acα-2,8Neu5Acα-2,3Galβ-1,3GalNAc β-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fucα-1,2Galβ-1,3GalNAcβ-1,4(Neu5Acα-2,3)Gal β-1,4Glc; all of which may be extended to the production of the corresponding gangliosides by reacting the above oligosaccharide moieties with ceramide or synthetizing the above oligosaccharides on a ceramide.

A ‘fucosylated oligosaccharide’ as used herein and as generally understood in the state of the art is an oligosaccharide that is carrying a fucose-residue. Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNF I), Lacto-N-fucopentaose II (LNF II), Lacto-N-fucopentaose III (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose.

A ‘neutral oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2′, 3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

A ‘fucosylation pathway’ as used herein is a biochemical pathway consisting of the enzymes and their respective genes, mannose-6-phosphate isomerase, phosphomannomutase, mannose-1-phosphate guanylyltransferase, GDP-mannose 4,6-dehydratase, GDP-L-fucose synthase and/or the salvage pathway L-fucokinase/GDP-fucose pyrophosphorylase, combined with a fucosyltransferase leading to α 1,2; α 1,3; α 1,4 or α 1,6 fucosylated oligosaccharides or fucosylated oligosaccharide containing bioproduct.

A ‘sialylation pathway’ is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, Glucosamine 6-phosphate N-acetyltransferase, N-AcetylGlucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, and/or CMP-sialic acid synthase, combined with a sialyltransferase leading to a 2,3; a 2,6 or a 2,8 sialylated oligosaccharides or sialylated oligosaccharide containing bioproduct.

A ‘galactosylation pathway’ as used herein is a biochemical pathway consisting of the enzymes and their respective genes, galactose-1-epimerase, galactokinase, glucokinase, galactose-1-phosphate uridylyltransferase, UDP-glucose 4-epimerase, glucose-1-phosphate uridylyltransferase, and/or glucophosphomutase, combined with a galactosyltransferase leading to an alpha or beta bound galactose on the 2, 3, 4, 6 hydroxyl group of a mono, di, oligo or polysaccharide containing bioproduct.

An ‘N-acetylglucosamine carbohydrate pathway’ as used herein is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridylyltransferase, glucosamine-1-phosphate acetyltransferase, and/or glucosamine-1-phosphate acetyltransferase, combined with a glycosyltransferase leading to an alpha or beta bound N-acetylglucosamine on the 3, 4, 6 hydroxyl group of a mono, di, oligo or polysaccharide containing bioproduct.

As used herein, the term “glycolipid” refers to any of the glycolipids, which are generally known in the art. Glycolipids (GLs) can be subclassified into Simple (SGLs) and Complex (CGLs) glycolipids. Simple GLs, sometimes called saccharolipids, are two-component (glycosyl and lipid moieties) GLs in which the glycosyl and lipid moieties are directly linked to each other. Examples of SGLs include glycosylated fatty acids, fatty alcohols, carotenoids, hopanoids, sterols or paraconic acids. Bacterially produced SGLs can be classified into rhamnolipids, glucolipids, trehalolipids, other glycosylated (non-trehalose containing) mycolates, trehalose-containing oligosaccharide lipids, glycosylated fatty alcohols, glycosylated macro-lactones and macro-lactams, glycomacrodiolides (glycosylated macrocyclic dilactones), glyco-carotenoids and glyco-terpenoids, and glycosylated hopanoids/sterols. Complex glycolipids (CGLs) are, however, structurally more heterogeneous, as they contain, in addition to the glycosyl and lipid moieties, other residues like, for example, glycerol (glycoglycerolipids), peptide (glycopeptidolipids), acylated-sphingosine (glycosphingolipids), or other residues (lipopolysaccharides, phenolic glycolipids, nucleoside lipids).

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, purity can be determined using methods such as but not limited to thin layer chromatography, gas chromatography, NMR, HPLC, capillary electrophoresis or mass spectroscopy.

The terms “precursor” as used herein refers to substances that are taken up or synthetized by the cell for the specific production of a bioproduct. In this sense a precursor can be an acceptor as defined herein, but can also be another substance, metabolite, which is first modified within the cell as part of the biochemical synthesis route of the product. Examples of such precursors comprise the acceptors as defined herein, and/or glucose, galactose, fructose, glycerol, sialic acid, fucose, mannose, maltose, sucrose, lactose glucose-1-phosphate, galactose-1-phosphate, UDP-glucose, UDP-galactose, glucose-6-phosphate, fructose-6-phosphate, fructose-1,6-bisphosphate, glycerol-3-phosphate, dihydroxyacetone, glyceraldehyde-3-phosphate, dihydroxyacetone-phosphate, glucosamine-6-phosphate, glucosamine, N-acetyl-glucosamine-6-phosphate, N-acetyl-glucosamine, N-acetyl-mannosamine, N-acetylmannosamine-6-phosphate, UDP-N-acetylglucosamine, N-acetylglucosamine-1-phosphate, N-acetylneuraminic acid (sialic acid), N-acetyl-Neuraminic acid—9 phosphate, CMP-sialic acid, mannose-6-phosphate, mannose-1-phosphate, GDP-mannose, GDP-4-dehydro-6-deoxy-α-D-mannose, and/or GDP-fucose.

The term “acceptor” as used herein refers to bioproducts that can be modified by, for example, but not limited to a sialyltransferase and/or fucosyltransferase and/or galactosyltransferase and/or N-acetylglucosamine transferase and/or N-acetylgalactosamine transferase and/or glucosyltransferase and/or mannosyltransferase and/or xylosyltransferase and/or oligosaccharyl-transferase complex and/or oligosaccharide-lipid mannosyltransferase. Examples of such acceptors are lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of, Lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.

### BRIEF DESCRIPTION OF THE DRAWINGS

The following drawings and examples will serve as further illustration and clarification of the disclosure and are not intended to be limiting.

FIG. 1 shows the relative specific growth rate of the wildtype and the GM strain when grown in minimal medium containing increasing concentrations of antibiotics, as a percentage of the maximum specific growth rate when no antibiotics are added (concentration range=0). The darker shaded bars represent the data for the WT strain while the lighter bars represent the data for the GM strain. Error bars are standard deviations calculated from at least three replicates.

FIG. 2 shows the relative specific growth rate of the wildtype and the GM strain when grown in minimal medium containing different concentrations of the osmolytes KCl, NaCl and sucrose. The darker shaded bars represent the data for the WT strain while the lighter bars represent the data for the GM strain. Error bars are standard deviations calculated from at least three replicates.

### DETAILED DESCRIPTION

The disclosure provides for a viable Gram-negative bacterial host cell wherein the cell comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPGs) and Glucosylglycerol, glycan, and trehalose.

In a preferred embodiment, the reduced or abolished synthesis is provided by a mutation in any one or more glycosyltransferase involved in the synthesis of any one of the poly-N-acetyl-glucosamine (PNAG), Enterobacterial Common Antigen (ECA), cellulose, colanic acid, core oligosaccharides, Osmoregulated Periplasmic Glucans (OPGs) and Glucosylglycerol, glycan, and trehalose. In another preferred embodiment, the mutation can alter the expression or the coding sequence of all non-essential or predicted non-essential glycosyltransferases. In a more preferred embodiment, the mutation provides for a deletion or lower expression of the glycosyltransferases. In a more preferred embodiment, the host cell is devoid of all non-essential glycosyltransferases. The cell is called a glycominimized cell.

According to the disclosure, the cell has mutation(s) in the expression or the coding sequence of any one or more of glycosyltransferase encoding genes. In some embodiments of the disclosure the mutation of the glycosyltransferase encoding gene completely knocks out the glycosyltransferase encoding gene to be obtained in ways as known by the person skilled in the art. In some embodiments of the disclosure the mutation of the glycosyltransferase encoding gene is i) a mutation that creates a premature stop codon in the glycosyltransferase encoding gene, ii) a mutation in the catalytic domain of the glycosyltransferase, iii) a mutation in the acceptor-binding domain of the glycosyltransferase, iv) a mutation in the glycan donor-binding domain of the glycosyltransferase, resulting in the same phenotype as a knock-out mutant. According to specific embodiments of the disclosure the reduced expression of the glycosyltransferase encoding gene comprises any one or more of: i) mutating the transcription unit of the glycosyltransferase encoding gene; ii) mutating the endogenous/homologous promoter of the glycosyltransferase encoding gene; iii) mutating the ribosome binding site of the glycosyltransferase encoding gene; iv) mutating an UTR of the glycosyltransferase encoding gene and/or v) mutating the transcription terminator.

Essential genes are those genes that are indispensable for the survival of an organism under certain conditions. Essential genes of an organism constitute its minimal gene set, which is the smallest possible group of genes that would be sufficient to sustain a functioning cellular life form under the most favorable conditions (Fang et al., Mol. Biol. Evol. (2005), 22(11), 2147-2156; Zhang and Lin, Nucleic Acids Res. (2009), 37, D455-458). As defined herein, a “non-essential and predicted non-essential glycosyltransferase” refers to a glycosyltransferase that is not critical for the host cell for its survival in rich growth media. Inactivation of the genes encoding for the glycosyltransferases and polypeptides predicted to be the glycosyltransferases from the host genome does not lead to a lethal phenotype of the host when grown in media wherein all necessary nutrients are provided.

The non-essential and predicted non-essential glycosyltransferase encoding genes encompass at least those genes encoding subunits of the poly-N-acetyl-D-glucosamine synthase, UDP-N-acetylglucosamine-undecaprenyl-phosphate N-acetylglucosaminephosphotransferase, Fuc4NAc (4-acetamido-4,6-dideoxy-D-galactose) transferase, UDP-N-acetyl-D-mannosaminuronic acid transferase, the catalytic subunits of the cellulose synthase, cellulose biosynthesis protein, colanic acid biosynthesis glucuronosyltransferase, colanic acid biosynthesis galactosyltransferase, colanic acid biosynthesis fucosyltransferase, UDP-glucose:undecaprenyl-phosphate glucose-1-phosphate transferase, putative colanic biosynthesis glycosyl transferase, UDP-glucuronate:LPS(HepIII) glycosyltransferase, ADP-heptose-LPS heptosyltransferase 2, ADP-heptose:LPS heptosyltransferase 1, putative ADP-heptose:LPS heptosyltransferase 4, lipopolysaccharide core biosynthesis protein, UDP-glucose:(glucosyl)LPS α-1,2-glucosyltransferase, UDP-D-glucose:(glucosyl)LPS α-1,3-glucosyltransferase, UDP-D-galactose:(glucosyl)lipopolysaccharide-1,6-D-galactosyltransferase, lipopolysaccharide glucosyltransferase I, lipopolysaccharide core heptosyltransferase 3, β-1,6-galactofuranosyltransferase, undecaprenyl-phosphate 4-deoxy-4-formamido-L-arabinose transferase, lipid IVA 4-amino-4-deoxy-L-arabinosyltransferase, bactoprenol glucosyl transferase, putative family 2 glycosyltransferase gene, osmoregulated periplasmic glucans (OPGs) biosynthesis protein G, OPG biosynthesis protein H, glucosylglycerate phosphorylase, glycogen synthase, 1,4-α-glucan branching enzyme, 4-α-glucanotransferase, trehalose-6-phosphate synthase, and preferably putative non-essential glycosyltransferases.

Any one or more of the mutations in any one or more of the glycosyltransferase genes encoding the subunits of the poly-N-acetyl-D-glucosamine synthase subunits will reduce and/or abolish the synthesis of PNAG. The synthesis of the Enterobacterial Common Antigen (ECA) will be reduced and/or abolished by any one or more of the mutations in any one or more of the glycosyltransferase genes encoding the UDP-N-acetylglucosamine-undecaprenyl-phosphate N-acetylglucosaminephosphotransferase, Fuc4NAc (4-acetamido-4,6-dideoxy-D-galactose) transferase or UDP-N-acetyl-D-mannosaminuronic acid transferase involved in ECA synthesis. In the same embodiment, the cellulose biosynthesis will be negatively affected by any one or more of the mutations in any one or both cellulose synthase catalytic subunits and/or the cellulose biosynthesis protein. In the same preferred embodiment, the synthesis of the exopolysaccharide colanic acid is reduced and/or abolished by any one or more of the mutations in any one or more of the glycosyltransferase encoding genes colanic acid biosynthesis glucuronosyltransferase, colanic acid biosynthesis galactosyltransferase, colanic acid biosynthesis fucosyltransferase, UDP-glucose: undecaprenyl-phosphate glucose-1-phosphate transferase and/or putative colanic biosynthesis glycosyl transferase. The synthesis of core oligosaccharides will be reduced and/or abolished by any one or more of the mutations in any one or more of the glycosyltransferase encoding genes UDP-glucuronate:LPS(HepIII) glycosyltransferase, ADP-heptose-LPS heptosyltransferase 2, ADP-heptose:LPS heptosyltransferase 1, putative ADP-heptose:LPS heptosyltransferase 4, lipopolysaccharide core biosynthesis protein, UDP-glucose:(glucosyl)LPS α-1,2-glucosyltransferase, UDP-D-glucose:(glucosyl)LPS α-1,3-glucosyltransferase, UDP-D-galactose:(glucosyl)lipopolysaccharide-1,6-D-galactosyltransferase, lipopolysaccharide glucosyltransferase I, lipopolysaccharide core heptosyltransferase 3, β-1,6-galactofuranosyltransferase, undecaprenyl-phosphate 4-deoxy-4-formamido-L-arabinose transferase, lipid IVA 4-amino-4-deoxy-L-arabinosyltransferase, bactoprenol glucosyl transferase, putative glycosyltransferases and/or the putative family 2 glycosyltransferase. The production of OPG will be negatively affected by any one or more of the mutations in any one or both glycosyltransferase encoding genes OPG biosynthesis protein G and/or OPG biosynthesis protein H. The production of Glucosylglycerol will be negatively affected by the mutations in the glucosylglycerate phosphorylase gene. In the same preferred embodiment the glycan synthesis will be negatively affected by any or more of the mutations in any one or more of the genes encoding the glycogen synthase, 1,4-α-glucan branching enzyme and/or 4-α-glucanotransferase. Trehalose synthesis will be reduced and/or abolished by any one or more of the mutations in the trehalose-6-phosphate synthase gene.

Alternatively or additionally, the PNAG synthesis can be reduced or abolished by any one or more of i) over-expression of a carbon storage regulator encoding gene, ii) deletion of a Na-/H antiporter regulator encoding gene or iii) deletion of the sensor histidine kinase encoding gene.

In a specific exemplary embodiment according to the disclosure the host cell's PNAG synthesis is reduced or abolished by mutation of the genes pgaC or pgaD, or the PNAG synthesis is reduced or abolished by any one or more of i) over-expression of the csrA encoding gene, ii) deletion of the regulator encoding gene NhaR or iii) deletion of the kinase encoding gene resC; the host cell's ECA synthesis is reduced or abolished by mutation of any one or more of the genes rfe, rffT or rffM, the host cell's cellulose synthesis is reduced or abolished by mutation of the genes bcsA, bcsB or bcsC, the host cell's colanic acid synthesis is reduced or abolished by mutation of any one or more of the genes wcaA, wcaC, wcaE, wcaI, wcaJ or wcaL, the host cell's core oligosaccharides synthesis is reduced or abolished by mutation of any one or more of the genes waaH, waaF, waaC, waaU, waaZ, waaJ, waaO, waaB, waaS, waaG, waaQ, wbbI, arnC, arnT, yaiP, yfdH or wbbK, the host cell's OPG and Glucosylglycerol synthesis is reduced or abolished by mutation of the genes opgG, opgH or ycjM, the host cell's glycan synthesis is reduced or abolished by mutation of any one or more of the genes glgA, glgB or malQ, and the host cell's trehalose synthesis is reduced or abolished by mutation of the otsA gene.

According to specific embodiments, the cell is characterized by at least one of: (a) not impairing growth or growth speed of the cells, (b) enhancing growth of growth speed of the cells, (c) not impairing biomass production in a fermentation using the cell, (d) enhancing biomass production in a fermentation using the cell, (e) reducing biomass production in a fermentation using the cell, (f) not impairing viscosity in a fermentation, (g) lowering viscosity in a fermentation, (h) not impairing biofilm formation in a fermentation, (i) reducing biofilm formation in a fermentation, (j) not impairing osmotic pressure in a fermentation or (k) improving osmotic pressure in a fermentation compared to a reference cell without reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPGs) and Glucosylglycerol, glycan, and trehalose. Each possibility represents a separate embodiment of the disclosure.

According to the disclosure the host cell belongs to the family of Gram-negative bacteria. The latter bacteria preferably belong to the phylum of the Proteobacteria. In another embodiment the host cell is selected from the group consisting of Escherichia spp., Shigella spp., Salmonella spp., Campylobacter spp., Neisseria spp., Moraxella spp., Stenotrophomonas spp., Bdellovibrio spp., Acinetobacter spp., Enterobacter spp., Haemophilus spp., Aeromonas spp., Francisella spp., Yersinia spp., Klebsiella spp., Bordetella spp., Legionella spp., Citrobacter spp., Chlamydia spp., Brucella spp., Pseudomonas spp., Helicobacter spp. and Vibrio spp. According to the disclosure, the host cell preferably belongs to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the disclosure specifically relates to a mutated and/or transformed Escherichia coli host cell or strain as indicated above wherein the E. coli strain is a K12 strain. More preferably, the Escherichia coli K12 strain is E. coli MG1655.

In another range of embodiments, the disclosure provides a viable transgenic cell genetically modified to produce at least one bioproduct as disclosed herein. The cell can grow and divide and has reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPGs) and Glucosylglycerol, glycan, and trehalose. The reduced or abolished synthesis preferably results from a mutation in the expression or the coding sequence of at least one gene encoding proteins involved in the production of PNAG, ECA, cellulose, colanic acid, core oligosaccharides, OPGs, Glucosylglycerol, glycan, trehalose. The genes are glycosyltransferase genes and the proteins are glycosyltransferases. In the most preferred embodiments, the host cell is devoid of all non-essential glycosyltransferases.

The disclosure also provides a method for producing at least one bioproduct as described herein with a bacterial cell as described herein. First, a host cell, which is genetically modified to produce at least one bioproduct as described herein is provided. Preferably, at least one non-essential glycosyltransferase encoding gene of the cell has been mutated and/or has a reduced expression. The cell is cultivated in a medium under conditions permissive for the production of the desired bioproduct. Preferably, the bioproduct is separated from the cultivation. More preferably, the bioproduct is purified after separation from the cultivation.

In a further specific exemplary embodiment, the disclosure provides a method for increasing the production of at least one bioproduct as described herein with an E. coli cell, which is genetically modified to have reduced or abolished synthesis of non-essential glycosyltransferases to produce at least one bioproduct as compared to an E. coli cell genetically modified to produce the bioproduct(s) but lacking the extra reduced expression and/or mutation described hereafter. An E. coli cell, which is genetically modified to produce at least one bioproduct is further altered by providing a mutation in and/or a reduced expression of an endogenous glycosyltransferase encoding gene. The cell is cultivated in a medium under conditions permissive for the production of the desired bioproduct. Preferably the bioproduct is separated from the cells and the cultivation. The term “separating” means harvesting, collecting or retrieving the bioproduct from the host cells and/or the medium of its growth via conventional methods. Conventional methods to free or to extract the bioproduct out of the cells can be used, such as cell destruction using high pH, heat shock, sonication, French press, homogenization, enzymatic hydrolysis, chemical hydrolysis, solvent hydrolysis, detergent, hydrolysis, . . . The culture medium and/or cell extract, collectively called the “bioproduct containing mixtures”, can then be further used for separating the bioproduct. This preferably involves clarifying the bioproduct containing mixtures to remove suspended particulates and contaminants, particularly cells, cell components, insoluble metabolites and debris produced by culturing the genetically modified cell. In this step, the bioproduct containing mixture can be clarified in a conventional manner. Preferably, the bioproduct containing mixture is clarified by centrifugation, flocculation, decantation and/or filtration. A second step of separating the bioproduct from the bioproduct containing mixture preferably involves removing substantially all the proteins, as well as peptides, amino acids, RNA and DNA and any endotoxins and glycolipids that could interfere with the subsequent separation step, from the bioproduct containing mixture, preferably after it has been clarified. In this step, proteins and related impurities can be removed from the bioproduct containing mixture in a conventional manner. Preferably, proteins, salts, by-products, color and other related impurities are removed from the bioproduct containing mixture by ultrafiltration, nanofiltration, reverse osmosis, microfiltration, activated charcoal or carbon treatment, tangential flow high-performance filtration, tangential flow ultrafiltration, affinity chromatography, ion exchange chromatography (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, while the bioproduct remains in the bioproduct containing mixture. The bioproduct is further separated from the reaction mixture and/or culture medium and/or cell with or without further purification steps by evaporation, lyophilization, crystallization, precipitation, and/or drying, spray drying. In an even further aspect, the disclosure also provides for a possible further purification of the bioproduct by, for example, use of (activated) charcoal or carbon, nanofiltration, ultrafiltration or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the product. Another purification step is to dry, spray dry or lyophilize the bioproduct.

According to specific embodiments, the cell further modified for the production of the desired bioproduct is characterized by at least one of: (a) not impairing bioproduct production, (b) enhancing bioproduct production, (c) not impairing productivity in a fermentation, (d) enhancing productivity in a fermentation, (e) not impairing growth or growth speed of the cells, (f) enhancing growth of growth speed of the cells, (g) not impairing biomass production in a fermentation using the cell, (h) enhancing biomass production in a fermentation using the cell, (i) reducing biomass production in a fermentation using the cell, (j) not impairing yield in a fermentation, (k) enhancing yield in a fermentation, (l) not impairing viscosity in a fermentation, (m) lowering viscosity in a fermentation, (n) not impairing biofilm formation in a fermentation, (o) reducing biofilm formation in a fermentation, (p) not impairing osmotic pressure in a fermentation; and/or (q) improving osmotic pressure in a fermentation, compared to a non-glycominimized cell similarly modified for the production of the same bioproduct. Each possibility represents a separate embodiment of the disclosure.

In some embodiments of the disclosure the mutation and/or reduced expression of the glycosyltransferase encoding gene confers unaffected bioproduct production wherein similar or the same levels of bioproduct are produced as is produced by a cell having the same genetic make-up but lacking the modified expression of the endogenous glycosyltransferase encoding gene. Preferably, the mutation and/or reduced expression of the glycosyltransferase encoding gene confers enhanced bioproduct formation in or by the cell wherein the cell produces more bioproduct in comparison to a cell having the same genetic make-up but lacking the mutation and/or reduced expression of the glycosyltransferase encoding gene. In some other embodiments of the disclosure the mutation and/or reduced expression of the glycosyltransferase encoding gene confers unaffected cell growth, or cell growth speed, productivity and/or biomass production wherein similar or the same levels of cell growth speed and/or biomass is produced as the cell growth speed, productivity and or biomass produced by a cell having the same genetic make-up but lacking the mutation and/or reduced expression of the glycosyltransferase encoding gene. Preferably, the mutation and/or reduced expression of the glycosyltransferase encoding gene confers enhanced cell growth speed, productivity and/or biomass production in or by the cell wherein the cell produces more biomass, has a higher productivity and/or has an enhanced cell growth speed in comparison to a cell having the same genetic make-up but lacking the mutation and/or reduced expression of the glycosyltransferase encoding gene.

In one embodiment the glycominimized Gram-negative cell is transformed with at least one heterologous gene to produce a sialic acid pathway or sialylation pathway, or fucosylation pathway or galactosylation pathway or N-acetylglucosamine carbohydrate pathway. This cell is transformed by introduction of a heterologous gene, genetic cassette or set of genes as described in the art. A further embodiment of the disclosure provides a method to produce a fucosylated, sialylated, galactosylated oligosaccharide, N-acetylglucosamine containing oligosaccharide, or sialic acid with a cell as described herein, respectively.

In one embodiment of the disclosure the methods as described herein are producing the bioproduct FLNT III, also known as fucosylated Lacto-N-neo-tetraose (LNnT) or Gal(b1-4)[Fuc(a1-3)]GlcNAc(b1-3)Gal(b1-4)Glc, also known as lacto-N-fucopentaose III or FLNP III or Le^{x}-lactose or Lewis-X pentasaccharide with a modified glycominimized E. coli strain.

In a further embodiment, the disclosure provides for the use of a cell as described herein for the production of a bioproduct, and preferably in the methods as described herein.

Further advantages follow from the specific embodiments, the examples and the attached drawings. It goes without saying that the abovementioned features and the features that are still to be explained below can be used not only in the respectively specified combinations, but also in other combinations or on their own, without departing from the scope of the disclosure.

The disclosure relates to the following specific embodiments:

1. A viable Gram-negative bacterial host cell characterized in that the host cell comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG) and Glucosylglycerol, glycan, and trehalose.

2. The cell of embodiment 1 wherein the reduced or abolished synthesis is provided by a mutation in any one or more glycosyltransferase involved in the synthesis of any one of the poly-N-acetyl-glucosamine (PNAG), Enterobacterial Common Antigen (ECA), cellulose, colanic acid, core oligosaccharides, Osmoregulated Periplasmic Glucans and Glucosylglycerol (OPG), glycan, and trehalose.

3. The cell of any one of embodiment 1 or 2 wherein the reduced or abolished synthesis is provided by a mutation in the expression or the coding sequence of all non-essential or predicted non-essential glycosyltransferases of the cell.

4. The cell according to embodiment 3, wherein the mutation in the expression or the coding sequence provides for a deletion or lower expression of the glycosyltransferases.

5. The host cell according to any one of embodiment 1 to 4, wherein the host cell is devoid of all non-essential glycosyltransferases.

6. The host cell of any one of embodiment 1 to 5, wherein
the PNAG synthesis is reduced or abolished by mutation in the expression or the coding sequence of any one or more of the glycosyltransferase genes encoding poly-N-acetyl-D-glucosamine synthase subunits, or the PNAG synthesis is reduced or abolished by any one or more of i) over-expression of a carbon storage regulator encoding gene, ii) deletion of a Na⁺/H⁺ antiporter regulator encoding gene or iii) deletion of the sensor histidine kinase encoding gene.
the ECA synthesis is reduced or abolished by mutation in the expression or the coding sequence of any one or more of the glycosyltransferase genes encoding UDP-N-acetylglucosamine-undecaprenyl-phosphate N-acetylglucosaminephosphotransferase, Fuc4NAc (4-acetamido-4,6-dideoxy-D-galactose) transferase or UDP-N-acetyl-D-mannosaminuronic acid transferase,
the cellulose synthesis is reduced or abolished by mutation in the expression or the coding sequence of any one or both glycosyltransferase genes encoding the cellulose synthase catalytic subunits or the cellulose biosynthesis protein,
the colanic acid synthesis is reduced or abolished by mutation in the expression or the coding sequence of any one or more of the glycosyltransferase genes encoding colanic acid biosynthesis glucuronosyltransferase, colanic acid biosynthesis galactosyltransferase, colanic acid biosynthesis fucosyltransferase, UDP-glucose:undecaprenyl-phosphate glucose-1-phosphate transferase or putative colanic biosynthesis glycosyl transferase,
the core oligosaccharides synthesis is reduced or abolished by mutation of any one or more of the glycosyltransferase genes encoding UDP-glucuronate:LPS(HepIII) glycosyltransferase, ADP-heptose-LPS heptosyltransferase 2, ADP-heptose:LPS heptosyltransferase 1, putative ADP-heptose:LPS heptosyltransferase 4, lipopolysaccharide core biosynthesis protein, UDP-glucose:(glucosyl)LPS α-1,2-glucosyltransferase, UDP-D-glucose:(glucosyl)LPS α-1,3-glucosyltransferase, UDP-D-galactose:(glucosyl)lipopolysaccharide-1,6-D-galactosyltransferase, lipopolysaccharide glucosyltransferase I, lipopolysaccharide core heptosyltransferase 3 or β-1,6-galactofuranosyltransferase, undecaprenyl-phosphate 4-deoxy-4-formamido-L-arabinose transferase, lipid IVA 4-amino-4-deoxy-L-arabinosyltransferase, bactoprenol glucosyl transferase, putative glycosyltransferases or putative family 2 glycosyltransferase,
the OPG and Glucosylglycerol synthesis is reduced or abolished by mutation of any one or more of the glycosyltransferase genes encoding the osmoregulated periplasmic glucans (OPG) biosynthesis protein G, OPG biosynthesis protein H or glucosylglycerate phosphorylase,
the glycan synthesis is reduced or abolished by mutation of any one or more of the glycosyltransferase genes encoding glycogen synthase, 1,4-α-glucan branching enzyme or 4-α-glucanotransferase,
the trehalose synthesis is reduced or abolished by mutation of the glycosyltransferase gene encoding trehalose-6-phosphate synthase.

7. The host cell of any one of embodiment 1 to 6, wherein the PNAG synthesis is reduced or abolished by mutation of the genespgaC or pgaD, or the PNAG synthesis is reduced or abolished by any one or more of i) over-expression of the csrA encoding gene, ii) deletion of the regulator encoding gene NhaR or iii) deletion of the kinase encoding gene resC, the ECA synthesis is reduced or abolished by mutation of any one or more of the genes rfe, rffT or rffM, the cellulose synthesis is reduced or abolished by mutation of the genes bcsA, bcsB or bcsC, the colanic acid synthesis is reduced or abolished by mutation of any one or more of the genes wcaA, wcaC, wcaE, wcaI, wcaJ or wcaL, the core oligosaccharides synthesis is reduced or abolished by mutation of any one or more of the genes waaH, waaF, waaC, waaU, waaZ, waaJ, waaO, waaB, waaS, waaG, waaQ, wbbI, arnC, arnT, yaiP, yfdH or wbbK, the OPG and Glucosylglycerol synthesis is reduced or abolished by mutation of the genes opgG, opgH or ycjM, the glycan synthesis is reduced or abolished by mutation of any one or more of the genes glgA, glgB or malQ, the trehalose synthesis is reduced or abolished by mutation of the otsA gene.

8. Host cell according to any one of embodiment 1 to 7, wherein the host cell is selected from the group consisting of Escherichia spp., Shigella spp., Salmonella spp., Campylobacter spp., Neisseria spp., Moraxella spp., Stenotrophomonas spp., Bdellovibrio spp., Acinetobacter spp., Enterobacter spp., Haemophilus spp., Aeromonas spp., Francisella spp., Yersinia spp., Klebsiella spp., Bordetella spp., Legionella spp., Citrobacter spp., Chlamydia spp., Brucella spp., Pseudomonas spp., Helicobacter spp. and Vibrio spp.

9. Host cell according to any one of embodiment 1 to 8, wherein the host cell is selected from Escherichia spp., Salmonella spp., and Pseudomonas spp.

10. Host cell according to any one of embodiment 1 to 9, wherein the host cell is E. coli.

11. Host cell according to any one of embodiment 1 to 10, selected from the group consisting of K-12 strain, W3110, MG1655, B/r, BL21, O157:h7, O42, 101-1,1180, 1357, 1412, 1520, 1827-70, 2362-75, 3431, 53638, 83972, 929-78, 98NK2, ABU 83972, B, B088, B171, B185, B354, B646, B7A, C, c7122, CFT073, DH1, DH5a, E110019, E128010, E74/68, E851/71, EAEC 042, EPECa11, EPECa12, EPECa14, ETEC, H10407, F11, F18+, FVEC1302, FVEC1412, GEMS_EPEC1, HB101, HT115, KO11, LF82, LT-41, LT-62, LT-68, MS107-1, MS119-7, MS124-1, MS 145-7, MS 79-2, MS 85-1, NCTC 86, Nissle 1917, NT:H19, NT:H40, NU14, O103:H2, O103:HNM, 0103:K+, O104:H12, O108:H25, O109:H9, O111H-, O111:H19, O111:H2, O111:H21, O11:LNM, O115:H-, O115:HMN, O115:K+, O119:H6, O119:UT, 0124:H40, O127a:H6, O127:H6, O128:H2, O131:H25, O136:H-, O139:H28 (strain E24377A/ETEC), O13:H11, O142:H6, O145:H-, O153:H21, O153:H7, O154:H9, O157:12, O157:H-, O157:H12, O157:H43, O157:H45, O157:H7 EDL933, O157:NM, O15:NM, O177:H11, O17:K52:H18 (strain UMN026/ExPEC), O180:H-, O1:K1/APEC, O26, O26:H-, O26:H11, O26:H11 K60, O26:NM, O41:H-, O45:K1 (strain S88/ExPEC), O51:H-, O55:H51, O55:H6, O55:H7, O5:H-, O6, O63:H6, O63:HNM, O6:K15:H31 (strain 536/UPEC), O7:K1 (strain IAI39/ExPEC), O8 (strain IAI1), O81 (strain ED1a), O84:H-, O86a:H34, O86a:H40, O90:H8, O91:H21, O9:H4 (strain HS), O9:H51, ONT:H-, ONT:H25, OP50, Orough:H12, Orough:H19, Orough:H34, Orough:H37, Orough:H9, OUT:H12, OUT:H45, OUT:H6, OUT:H7, OUT:HNM, OUT:NM, RN587/1, RS218, 55989/EAEC, B/BL21, B/BL21-DE3, SE11, SMS-3-5/SECEC, UTI89/UPEC, TA004, TA155, TX1999, and Vir68.

12. Host cell according to any one of embodiment 1 to 11, wherein the cell is further transformed with one or more genes of interest operably linked to a promoter and/or UTR.

13. Host cell according to any one of embodiment 1 to 12, wherein the cell is genetically modified to produce at least one bioproduct.

14. Host cell according to embodiment 12 or 13, wherein the gene of interest is on a plasmid or chromosome and is expressed in the host cell.

15. Isolated host cell according to any one of embodiment 1 to 14.

16. A method for the production of a bioproduct using a genetically modified host cell, the method comprising the steps of:
providing a host cell, which has been genetically modified, such, that at least the cell is able to produce the bioproduct wherein the unmodified host cell is not able to produce the bioproduct, due to the introduction of at least one heterologous gene, encoding the bioproduct or an intermediate thereof, which is expressed in the host cell;
cultivating and/or growing the genetically modified host cell in a cultivation medium enabling to production of the bioproduct thereby producing the bioproduct obtainable from the medium the host cell is cultivated in;

characterized in that the host cell is a bacterial host cell according to any one of embodiment 1 to 14.

17. Use of the bacterial host cell of any one of embodiment 1 to 15 for the production of a bioproduct.

18. Use according to embodiment 17, wherein the bioproduct is glycosylated product, preferably a glycolipid, a glycoprotein or oligosaccharide.

19. Use according to embodiment 17 or 18, wherein the bioproduct is an oligosaccharide, preferably a mammalian milk oligosaccharide, more preferably chosen from the list of 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated milk oligosaccharides, neutral milk oligosaccharide and/or sialylated milk oligosaccharides, FLNT III.

20. Method according to embodiment 16, wherein the bioproduct is glycosylated product, preferably a glycolipid, a glycoprotein or oligosaccharide.

21. Method according to embodiment 16 or 20, wherein the bioproduct is an oligosaccharide, preferably a mammalian milk oligosaccharide, more preferably chosen from the list of 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated milk oligosaccharides, neutral milk oligosaccharide and/or sialylated milk oligosaccharides, FLNT III.

### EXAMPLES

### Example 1: Materials and Methods Escherichia coli

Media

The Luria Broth (LB) medium consisted of 1% tryptone (Becton Dickinson, Erembodegem, Belgium), 0.5% yeast extract (Becton Dickinson) and 0.5% sodium chlorate (VWR, Leuven, Belgium). Lysogeny broth agar (LBA) is similarly composed with the addition of 12 g/L agar (Sigma-Aldrich, Overijse, Belgium). The minimal medium for the growth experiments contained 2.00 g/L NH4Cl, 5.00 g/L (NH4)2SO4, 3.00 g/L KH2PO4, 7.3 g/L K2HPO4, 8.4 g/L MOPS, 0.5 g/L NaCl, 0.5 g/L MgSO4.7H2O, and 16.5 g/L glucose H2O, 1 ml/L vitamin solution and 100 μL/L molybdate solution. The vitamin solution contained 3.6 g/L FeCl2 4H2O, 5 g/L CaCl2.2H2O, 1.3 g/L MnCl2.2H2O, 0.38 g/L CuCl2.2H2O, 0.5 g/L CoCl2.6H2O, 0.94 g/L ZnCl2, 0.0311 g/L H3BO4, 0.4 g/L Na2EDTA 2H2O and 1.01 g/L thiamine HCl. The molybdate solution contained 0.967 g/L Na2MoO4.2H2O. Glucose was procured from Honeywell Riedel-De-Haën (Leuven, Belgium). All other chemicals were purchased from Sigma-Aldrich, unless stated otherwise.

The batch medium for fermentations contained 0.45 g/L (NH4)2SO4, 2.105 g/L KH2PO4, 1.68 g/L K2HPO4, 0.34 g/L NaCl, 1 g/L citric acid monohydrate, 1 g/L MgSO4 7H2O, 50 g/L glycerol, 50 g/L lactose.H2O, 9 mL of the vitamin solution and 0.9 mL of the molybdate solution with the same composition as described above. The fed-batch medium contained 1.9 g/L (NH4)2SO4, 3.05 g/L K2HPO4, 1.8 g/L (NH4)2HPO4, 4.5 g/L NH4H2PO4, 1 g/L citric acid monohydrate, 1 g/L MgSO4 7H2O, 250 g/L glycerol, 50 g/L lactose.H2O, 53 mL of the vitamin solution and 5 mL of the molybdate solution with the same composition as described above.

Complex medium was sterilized by autoclaving (121° C., 21′) and minimal medium by filtration (0.22 μm Sartorius). When necessary, the medium was made selective by adding antibiotics: e.g., ampicillin (100 μg/mL), spectinomycin (100 μg/μL), kanamycin (50 g/mL), gentamycin (30 μg/mL), chloramphenicol (34 μg/mL) or tetracyline (10 μg/mL).

Plasmids

Plasmids were constructed using CPEC according to Tian and Quan (PLoS One 4 (2009), e6441). Serine integrase attachment sites were integrated in the oligonucleotides.

The pLP1 vector (pJET-attB‘TT’-chlR-attP‘TT’) containing the chloramphenicol resistance gene flanked with the serine integrase attachment sites attB and attP with a ‘TT’ dinucleotide core sequence was constructed using the CloneJET PCR Cloning Kit (ThermoFisher, USA). The pLP6 vector (pJET-attB‘CA’-kanR-attP‘CA’) was constructed using the pLP1 vector as template in a CPEC reaction to contain a kanamycin resistance gene flanked with the serine integrase attachment sites attB and attP with a ‘CA’ dinucleotide core sequence. The plnt1 vector (with a temperature sensitive pSC101 origin of replication, ampicillin resistant) encoding the serine integrase PhiC31 was created using CPEC. The latter sequence was obtained from dr. Maria R. Foulquie-Moreno (VIB-KU Leuven Center for Microbiology, Belgium). The pTKRED vector was obtained from Edward Cox and Thomas Kuhlman (Nucleic Acids Res. 38 (2010), e92; Addgene plasmid #41062). Plasmids were maintained in the host E. coli Top10 (F-, mcrA, Δ(mrr-hsdRMS-mcrBC), Φ80lacZΔM15, ΔlacX74, recA1, araD139, Δ(araleu)7697, galU, galK, rpsL, (StrR), endA1, nupG) bought from Life Technologies.

Strains and Mutations

Escherichia coli K12 MG1655 [lambda-, F-, rph-1] was obtained from the Coli Genetic Stock Center (US), CGSC Strain #: 7740, in March 2007. Gene replacements were performed with knock-out cassettes using the SIRE technique adapted from Snoeck et al. (Biotechnol. Bioeng. 116 (2019), 364-374). This technique is based on homologous recombination as described by Kuhlman and Cox (Nucleic Acids Res. 38 (2010), e92) and antibiotic selection after site-directed integration performed by the PhiC31 serine integrase. Two types of knock-out cassettes were created: a first type of cassettes containing the selectable marker chloramphenicol flanked with attB and attP sites having the ‘TT’ dinucleotide core sequence and flanked by 100 bp homologies of a specific target operon for knock-out and a second type of cassettes containing the selectable marker kanamycin flanked with attB and attP sites having the ‘CA’ dinucleotide core sequence and flanked by 100 bp homologies of another specific target operon for knock-out. The knock-out cassettes were PCR amplified from template plasmids and transformed as linear DNA by electroporation. Cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600 Ω, 25 μFD, and 250 volts). Since both knock-out cassettes contained 2 different selection markers that were flanked with different att sites, which were orthogonal and did not interfere with each other, 2 operons could be knocked out successively and both selection markers could be recovered during one integration step as follows. A chloramphenicol-containing knock-out cassette was first introduced at a selected operon using homologous recombination. Selected colonies were subsequently prepared for another round of transformation where homologous recombination was performed at a second selected operon using a kanamycin selectable knock-out cassette. Next, selected colonies were prepared for the final transformation round in which the pInt1 plasmid containing the PhiC31 integrase was introduced. Cells containing the pInt1 plasmid were selected on spectinomycin while simultaneously expressing the integrase overnight with 0.4 mM isopropyl-β-D-thiogalactopyranoside induction on LBA plates. Colonies were checked by PCR to evaluate the removal of both landing pads, which was then confirmed using Sanger sequencing (LCG Genomics, Germany). The final strain containing a knock-out for both selected operons was cured of any remaining plasmids after overnight culturing at 42° C. and prepared for next transformation rounds for next operon knock-outs.

All strains are stored in cryovials at −80° C. (overnight LB culture mixed in a 1:1 ratio with 70% glycerol).

Heterologous and Homologous Expression

Genes that needed to be expressed, be it from a plasmid or from the genome were synthetically synthetized with one of the following companies: DNA2.0, Gen9, Twist Biosciences or IDT. Expression could be further facilitated by optimizing the codon usage to the codon usage of the expression host. Genes were optimized using the tools of the supplier. All promoters and UTRs originate from the libraries described by De Mey et al. (BMC Biotechnology, 2007) and Mutalik et al. (Nat. Methods 2013, No. 10, 354-360). Gene integrations were performed similarly as described above using the SIRE technique.

Cultivation Conditions

Cells were cultured in Greiner Bio-One (Vilvoorde, Belgium) polystyrene F-bottom 96 well plates using the defined medium described above and inoculated 1% from precultures, which were grown in a carbon limiting 2.2 g/L glucose H2O defined medium variant. For the minimum inhibitory concentration (MIC) determination and osmotic stress sensitivity experiments, antibiotics resp. salts were supplemented to the medium in a 100 mL volumetric flask and subsequently filter sterilized using a 0,22 m Corning filter system. Each strain was grown in multiple wells of the 96-well plate as biological replicates. These final 96-well culture plates were then incubated at 30° C. on an orbital shaker at 800 rpm for 72h, or longer

Fermentations were done in 5 L Biostat reactors (Sartorius Stedim Biotech, Germany). Temperature was kept at 37° C. using the water jacket. pH was maintained at 7.0 using a 98% H2SO4 and 25% NH40H solution. Aeration is kept at 1 L air/min and introduced in the reactor using a sparger after passing through a 0.2 m PTFE filter (Sartorius Stedim Biotech Midisart 2000, Germany). Off gas is cooled using an off-gas cooler, filtered and analyzed to detect the fraction CO2 and O2, respectively, by infrared and paramagnetic detection (ABB Automation EL3020, Germany). Foaming is suppressed using anti-foam Struktol J637 (Schill und Seilacher, Germany). Temperature, pH, pO2, added volume acid and base, added volume of medium are continuously monitored using Sartorius MFCS software.

The bioreactor containing 2 L physiological saline solution is subsequently sterilized by autoclaving for 1 hour at 121° C. and 1 atm overpressure. Next the solution is replaced by adding the above-mentioned batch medium and inoculated through the available septum with 100 mL preculture (defined medium) using sterile syringes. Fed batch medium is added using a peristaltic pump when the glycerol was completely consumed at a constant volumetric feeding rate.

Optical Density

Cell density of the cultures was frequently monitored by measuring optical density at 600 nm (Implen Nanophotometer NP80, Westburg, Belgium) or with a Spark 1OM microplate reader (Tecan, Switzerland).

Growth Rate Determination

The maximal growth rate (Max) was calculated based on the observed optical densities at 600 nm using the R package grofit.

MIC Determination

The minimum inhibitory concentration (MIC) was determined as the lowest concentration of an antibiotic where the growth rate was less or equal than 10% of the growth rate when no antibiotic was added.

Osmotic Stress Sensitivity Assay

To test the osmotic stress sensitivity of an E. coli strain, a concentration array of KCl, NaCl and sucrose was applied, and growth of the strain recorded. The growth rate was calculated and plotted in function of the concentration of each solute as well as of the osmotic pressure. The osmotic pressure π is calculated as π=i*C*R*T whereby i is the dimensionless Van't Hoff index, C the concentration of the solute (mol/L), R the ideal gas constant and T the temperature (K). For salts that dissociate in water, the Van't Hoff index is the sum of all ions originating from one molecule.

NGS Sequencing

gDNA of the glycominimized strain was extracted from 2 ml cell pellets using the Qiagen DNeasy Blood and Tissue kit (Qiagen) according to the manufacturer's recommendations. gDNA was eluted using nuclease-free water. The DNA concentration was determined via the Quant-iT™ PicoGreen dsDNA kit (Invitrogen). Standard genomic library preparation and Illumina HiSeq sequencing was performed by Fasteris (Geneva, Switzerland) using paired reads of 150 bp with an average coverage of 860. The NGS data quality was verified using FastQC (Babraham Informatics). Illumina adapters and low quality bases were removed using Trimmomatic v0.36 ((Bolger et al., Bioinformatics 30 (2014), 2114-2120). The trimmed data was analyzed using Breseq (Deatherage and Barrick, Methods Mol. Biol. 1151 (2014), pp. 165-188).

Liquid Chromatography

Samples from the growth experiments were analyzed with ThermoFisher's Exactive Plus Orbitrap Mass Spectrometer UPLC-MS in negative mode. Samples from the bioreactor experiments were pelleted and filtered (0.2 m) prior to analysis with the Waters Acquity H-class UPLC-ELSD system. Fed-batch samples were diluted 1:1 with 200 g/L trichloro-acetic acid to precipitate proteins and cell debris and are subsequently pelleted and filtered (0.2 μm). The sugars are separated using the Waters Acquity BEH Amide Column (130 Å, 1.7 μm, 2.1 mm×100 mm) and an isocratic eluent (75% acetonitrile, 0.15% triethylamine) at 0.6 mL/min and 35° C. Fed-batch samples for organic acid analysis were diluted 1:1 with methanol to precipitate proteins and cell debris and are subsequently pelleted and filtered (0.2 μm). The organic acids were separated using the Phenomenex Rezex ROA-H+column (8 m 4.6 mm×100 mm) and an isocratic eluent (10 mM H2SO4) at 0.1 mL/min and 40° C. and analyzed with the Waters Acquity H-class UPLC-UV system.

Calculations of Fermentation Parameters

The specific productivity Qp is the specific production rate of the oligosaccharide product, typically expressed in mass units of product per mass unit of biomass per time unit (=g oligosaccharide/g biomass/h). The Qp value has been determined for each phase of the fermentation runs, i.e., Batch and Fed-Batch phase, by measuring both the amount of product and biomass formed at the end of each phase and the time frame each phase lasted.

The specific productivity Qs is the specific consumption rate of the substrate typically expressed in mass units of substrate per mass unit of biomass per time unit (=g substrate/g biomass/h). The Qs value has been determined for each phase of the fermentation runs, i.e., Batch and Fed-Batch phase, by measuring both the total amount of substrate consumed and biomass formed at the end of each phase and the time frame each phase lasted.

The yield on substrate Ys is the fraction of product that is made from substrate and is typically expressed in mass unit of product per mass unit of substrate (=g oligosaccharide/g substrate). The Ys has been determined for each phase of the fermentation runs, i.e., Batch and Fed-Batch phase, by measuring both the total amount of oligosaccharide produced and total amount of substrate consumed at the end of each phase.

The yield on biomass Yx is the fraction of biomass that is made from substrate and is typically expressed in mass unit of biomass per mass unit of substrate (=g biomass/g substrate). The Yp has been determined for each phase of the fermentation runs, i.e., Batch and Fed-Batch phase, by measuring both the total amount of biomass produced and total amount of substrate consumed at the end of each phase.

The rate is the speed by which the product is made in a fermentation run, typically expressed in concentration of product made per time unit (=g oligosaccharide/L/h).

The rate is determined by measuring the concentration of oligosaccharide that has been made at the end of the Fed-Batch phase and dividing this concentration by the total fermentation time.

### Example 2: Creation of the Glycominimized E. coli Strain Lacking 38 Non-Essential Glycosyltransferase Genes

Starting from the wild-type E. coli K-12 MG1655 strain a glycominimized (GM) E. coli strain was created in which 38 non-essential glycosyltransferase genes were deleted. These genes included

1) the glycosyltransferase genes involved in the synthesis of PNAG, i.e., pgaC encoding for the poly-N-acetyl-D-glucosamine synthase subunit pgaC and pgaD encoding for the poly-N-acetyl-D-glucosamine synthase subunit pgaD,

2) the glycosyltransferase genes involved in the synthesis of ECA, i.e., rfe encoding for the UDP-N-acetylglucosamine-undecaprenyl-phosphate N-acetylglucosaminephosphotransferase, rffT encoding for the Fuc4NAc (4-acetamido-4,6-dideoxy-D-galactose) transferase and rffM encoding for the UDP-N-acetyl-D-mannosaminuronic acid transferase,

3) the glycosyltransferase genes involved in the synthesis of cellulose, i.e., bcsA encoding for the cellulose synthase catalytic subunit, bcsB encoding for the cellulose synthase periplasmic subunit and bcsC encoding for the cellulose biosynthesis protein,

4) the glycosyltransferase genes involved in the synthesis of colanic acid, i.e., wcaA encoding for the colanic acid biosynthesis glucuronosyltransferase, wcaC encoding for the colanic acid biosynthesis galactosyltransferase, wcaE encoding for the colanic acid biosynthesis fucosyltransferase, wcaI encoding for the colanic acid biosynthesis fucosyltransferase, wcaJ encoding for the UDP-glucose:undecaprenyl-phosphate glucose-1-phosphate transferase and wcaL encoding for a putative colanic biosynthesis glycosyl transferase,

5) the glycosyltransferase genes involved in the synthesis of core oligosaccharides that are attached to lipid A, i.e., waaH encoding for the UDP-glucuronate:LPS(HepIII) glycosyltransferase, waaF encoding for the ADP-heptose-LPS heptosyltransferase 2, waaC encoding for the ADP-heptose:LPS heptosyltransferase 1, waaU encoding for a putative ADP-heptose:LPS heptosyltransferase 4, waaZ encoding for a lipopolysaccharide core biosynthesis protein, waaJ encoding for the UDP-glucose:(glucosyl)LPS α-1,2-glucosyltransferase, waaO encoding for the UDP-D-glucose:(glucosyl)LPS α-1,3-glucosyltransferase, waaB encoding for the UDP-D-galactose:(glucosyl)lipopolysaccharide-1,6-D-galactosyltransferase, waaS encoding for the lipopolysaccharide core biosynthesis protein, waaG encoding for the lipopolysaccharide glucosyltransferase I, waaQ encoding for the lipopolysaccharide core heptosyltransferase 3, wbbI encoding for the β-1,6-galactofuranosyltransferase, arnC encoding for the undecaprenyl-phosphate 4-deoxy-4-formamido-L-arabinose transferase, arnT encoding for the lipid IVA 4-amino-4-deoxy-L-arabinosyltransferase, yfdH encoding for the bactoprenol glucosyl transferase and wbbK encoding for a putative glycosyltransferase,

6) the glycosyltransferase genes involved in the synthesis of OPGs and Glucosylglycerol, i.e., opgG encoding for the osmoregulated periplasmic glucans biosynthesis protein G and opgH encoding for the osmoregulated periplasmic glucans biosynthesis protein H and ycjM encoding for the glucosylglycerate phosphorylase

7) the glycosyltransferase genes involved in glycan synthesis, i.e., glgA encoding for the ADPglucose:1,4-α-D-glucan 4-α-D-glucosyltransferase, glgB encoding for the 1,4-α-glucan branching enzyme and malQ encoding for the 4-α-glucanotransferase,

8) the glycosyltransferase gene involved in trehalose synthesis, i.e., otsA encoding for the trehalose-6-phosphate synthase,

9) a predicted glycosyltransferase gene yaiP encoding for the putative family 2 glycosyltransferase.

The genome of the thus obtained glycominimized (GM) strain was sequence verified using next-generation sequencing that confirmed the presence of all envisaged knock-outs (and leaving a 53 bp attL scar at each operon removed) resulting in a modified strain of which about 100 kb or more than 2% of the entire genome has been deleted compared to the genome of its wildtype counterpart.

### Example 3: Creation of a Glycominimized E. coli Strain

In an alternative experiment, the synthesis of PNAG in an E. coli strain that already obtained a reduced or abolished synthesis of ECA, cellulose, colanic acid, core oligosaccharides, osmoregulated periplasmic glucans and Glucosylglycerol, glycan and trehalose can also be reduced or abolished by any one or more of the following mutations, i.e., by over-expression of the csrA gene, by deletion of the regulator encoding gene NhaR or by deletion of the kinase encoding gene rcsC.

### Example 4: Evaluation of the Glycominimized E. coli Strain for Growth and Sensitivity Toward Antibiotics and Osmotic Stress

The glycominimized (GM) strain as created in Example 2 was evaluated for growth in minimal medium lacking any antibiotics according to the cultivation conditions provided in Example 1. The maximal growth speed measured for the GM strain lacking 38 non-essential glycosyltransferase encoding genes was about 90% of the wildtype's (WT) growth speed.

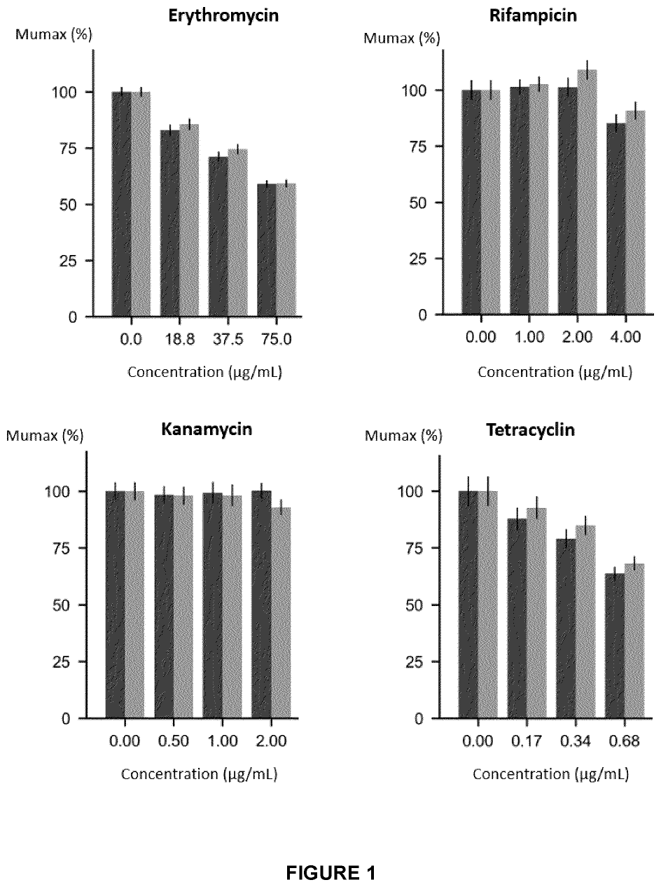

Since the cell wall of the GM strain has undergone a major transformation due to the loss of the O-antigen, core oligosaccharides and several exopolysaccharides, a next experiment was set up to the test the sensitivity of the GM toward commonly used antibiotics. In this experiment, the growth rate was measured for both the wildtype and the GM strain when grown in media containing increasing antibiotic concentrations. Compared to the WT strain, the GM strain showed to be similarly sensitive to erythromycin, rifampicin, kanamycin and tetracycline (FIG. 1). Also, similar minimum inhibitory concentrations (MIC) for those antibiotics were obtained for both strains.

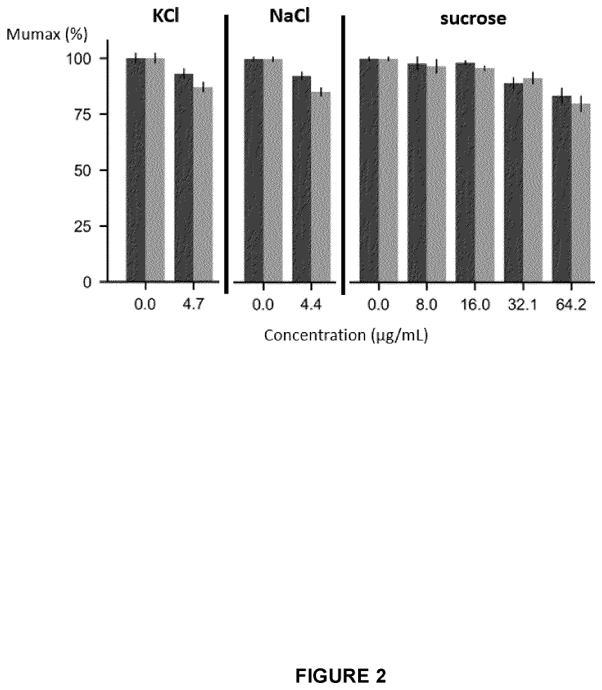

Since the semi-permeable membrane of the GM strain is severely altered compared to that of the WT strain, the osmotic sensitivity of both strains was compared toward KCl, NaCl and sucrose. FIG. 2 shows the GM strain is equally sensitive as the WT strain at 5 g/L concentrations of the salt solutes and up till 64 g/L for sucrose.

### Example 5: Fermentation of the Glycominimized E. coli Strain

In a next experiment, the GM strain can be evaluated in fed-batch fermentations at bioreactor scale, as described in Example 1. The strain's performance in the bioreactor will be similar or better compared to the reference strains in any of the following parameters: substrate uptake/conversion rate Qs (g substrate/g Biomass/h), product purity, growth speed, antifoam addition, viscosity, fermentation time.

### Example 6: Creation of an GM E. coli Strain with Elevated Production of Nucleotide-Activated Sugars

In a next experiment, the GM strain created in Example 2 was further engineered enabling the strain to produce more nucleotide-activated sugars compared to the wild-type E. coli. In a first part, the availability of the activated donor sugar UDP-GlcNAc was enhanced by knocking out the glucosamine-6-P deaminase encoding gene nagB. Next, the availability of UDP-galactose was enhanced by knocking out the UDP-sugar hydrolase encoding gene ushA and the galactose-1-P uridylyltransferase encoding gene galT. To increase the flux toward GDP-fucose and because the complete colanic acid operon including the genes cpsB, cpsG, gmd and fcl are deleted in the GM strain created in Example 2, a constitutive expression construct consisting of cpsB, cpsG, gmd and fcl was cloned and integrated in the host's genome as described in Example 1. The new GM production strain thus created can be evaluated in fed-batch fermentations at bioreactor scale, as described in Example 1. The strain's performance in the bioreactor is better compared to the reference GM strain lacking the nagB KO, the ushA KO, the galT KO and the KI construct with cpsB, cpsG, gmd and fcl in any of the following parameters: product titer, substrate uptake/conversion rate Qs (g substrate/g Biomass/h), product purity, growth speed, antifoam addition, viscosity, fermentation time.

### Example 7: Creation of an GM E. coli Strain that is Able to Produce Fucosyllacto-N-Neotetraose III (FLNT III)

In another example, the GM strain created in Example 6 was further engineered enabling the strain to produce fucosyllacto-N-neotetraose III (FLNT III). Lactose degradation was eliminated while maintaining lactose import by a knockout of the lacZYA operon and a knockin of the lactose permease encoding gene lacY under a constitutive promoter on the same locus. For FLNT III synthesis, knock-ins of constitutive expression constructs containing a galactoside beta-1,3-N-acetylglucosaminyltransferase (lgtA) and a beta-1,4-galactosyltransferase (lgtB), both from Neisseria meningitidis, were introduced together with an alpha-1,3-fucosyltransferase. Four alpha-1,3-fucosyltransferases were analyzed for FLNT III production: originating from Helicobacter pylori NCTC11639, Bacteroides fragilis NCTC9343, Helicobacter hepaticus ATCC51449 and from Helicobacter pylori UA948. In order to have a reference strain, the wildtype E. coli K12 MG1655 strain was similarly adapted for FLNT III production with KOs for nagB, ushA, galT, lacZYA and with KIs for lacY, lgtA, lgtB, cpsG, cpsB, gmd,fcl and an alpha-1,3-fucosyltransferase.

All mutant strains, both with the wildtype background as with the GM background, were evaluated and proven to produce FLNT III in a growth experiment as described in Example 1. Independent from the alpha-1,3-fucosyltransferase expressed, the GM production strains always had elevated GDP-fucose fluxes toward product compared to the reference strains and produced higher titers of FLNT III than the reference strains (see Table 1).

TABLE 1
Production of FLNT III normalized over OD600 (mg/L ·
OD600) in mutant production strains with a
wildtype (WT) or glycominimized (GM) background,
each time expressing another alpha-1,3-fucosyltransferase

| | Host (WT | Host (GM |
|---|---|---|
| Origin of the alpha-1,3-fucosyltransferase | background) | background) |
| Helicobacter pylori NCTC11639 | 11 | 48 |
| Bacteroides fragilis NCTC9343 | 10 | 14 |
| Helicobacter hepaticus ATCC51449 | 33 | 58 |
| Helicobacter pylori UA948 | 14 | 28 |

The FLNT III production strains created with the alpha-1,3-fucosyltransferase from Helicobacter hepaticus ATCC51449 were further evaluated for FLNT III production in fed-batch fermentations at bioreactor scale as described in Example 1. Both production strains (with WT and GM background) demonstrated to have a similar maximal growth rate and reached similar FLNT III production titers and yield, but the GM background strain had 20h shortened fermentation time with less undesired acid produced like succinate and acetate compared to the WT strain with the same FLNT III pathway introduced. The GM strain also showed higher production rates for the intermediate compounds LN3 and LNnT compared to the reference strain. The fermentation run of the FLNT III production strain with the GM background also displayed better performance for antifoam addition and viscosity compared to the fermentation run of the reference FLNT III production strain.

### Examples 8: Production of Phosphorylated and/or Activated Monosaccharides in an GM E. coli Strain

The GM E. coli strains as created in Example 2 and Example 3 can be used for the production of phosphorylated and/or activated monosaccharides. Examples of phosphorylated monosaccharides include but are not limited to glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some but not all of these phosphorylated monosaccharides are precursors or intermediates for the production of activated monosaccharide. Examples of activated monosaccharides include but are not limited to GDP-fucose, UDP-glucose, UDP-galactose and UDP-N-acetylglucosamine. These phosphorylated monosaccharides and/or activated monosaccharides can be produced in higher amounts than naturally occurring in E. coli e.g., by introducing some of the genetic modifications as described in Example 1. An E. coli strain with active expression units of the sucrose phosphorylase and fructokinase genes is able to grow on sucrose as a carbon source and can produce high(er) amounts of glucose-1P, as described in WO2012/007481. Such a strain additionally containing a knock-out of the genes pgi, pfkA and pfkB accumulate fructose-6-phosphate in the medium when grown on sucrose. Alternatively, by knocking out genes coding for (a) phosphatase(s) (agp), glucose 6-phosphate-1-dehydrogenase (zwf), phosphoglucose isomerase (pgi), glucose-1-phosphate adenylyltransferase (glgC), phosphoglucomutase (pgm) a mutant is constructed, which accumulates glucose-6-phosphate.

Alternatively, the strain according to the disclosure and further containing a sucrose phosphorylase and fructokinase with an additional overexpression of the wild type or variant protein of the L-glutamine-D-fructose-6-phosphate aminotransferase (glmS) from E. coli can produce higher amounts of glucosamine-6P, glucosamine-1P and/or UDP-N-acetylglucosamine. The GM strain will already have an increased pool of GDP-fucose by the knockout of the wcaJ coding for the UDP-glucose:undecaprenyl-phosphate glucose-1-phosphate transferase. An increased pool of UDP-glucose and/or UDP-galactose could be achieved by overexpressing the E. coli enzymes glucose-1-phosphate uridyltransferase (galU) and/or UDP-galactose-4-epimerase (galF). Alternatively, by overexpressing genes coding for galactokinase (galK) and galactose-1-phosphate uridylyltransferase (for example, originating from Bifidobacterium bifidum) the formation of UDP-galactose is enhanced by additionally knocking out genes coding for (a) phosphatase(s) (agp), UDP-glucose, galactose-1P uridylyltransferase (galT), UDP-glucose-4-epimerase (galE) a mutant is constructed, which accumulates galactose-1-phosphate

Another example of an activated monosaccharide is CMP-sialic acid, which is not naturally produced by E. coli. Production of CMP-sialic acid can e.g., be achieved by knocking out the nagAB operon encoding for the N-acetylglucosamine-6-phosphate deacetylase (nagA) and the glucosamine-6-phosphate deaminase (nagB) genes and further introducing next to a knock-in of glmS also a knock-in for the glucosamine-6-P-aminotransferase from S. cerevisiae (ScGNA1), an N-acetylglucosamine-2-epimerase from Bacteroides ovatus (BoAGE) and a sialic acid synthase from Campylobacter jejuni (CjneuB) and a CMP-sialic acid synthetase from Neisseria meningitidis (NmneuA).

Such strains can be used in a bio-fermentation process to produce these phosphorylated monosaccharides or activated monosaccharides in which the strains are grown on e.g., one or more of the following carbon sources: sucrose, glucose, glycerol, fructose, lactose, arabinose, maltotriose, sorbitol, xylose, rhamnose and mannose.

### Example 9: Production of Monosaccharides or Disaccharides in an GM E. coli Strain

The GM E. coli strains as created in Example 2 and Example 3 can be used for the production of monosaccharides or disaccharides. An example of such a monosaccharide is L-fucose. An GM E. coli fucose production strain can be created e.g., by starting from a GM strain that is able to produce FLNT III as described in Example 7 and by additionally knocking out the E. coli genes fucK and fucI (coding for an L-fucose isomerase and an L-fuculokinase) to avoid fucose degradation, and by expressing an 1,3-alpha-L-fucosidase (e.g., afcB from Bifidobacterium bifidum) to degrade FLNT III into fucose and LNnT. Such a strain can be used in a bio-fermentation process to produce L-fucose in which the strain is grown on sucrose, glucose or glycerol and in the presence of catalytic amounts of LNnT as an acceptor substrate for the alpha-1,3-fucosyltransferase. An example of such a disaccharide is e.g., lactose (galactose-beta,1,4-glucose). An GM E. coli lactose production strain can be created e.g., by introducing in the GM E. coli strain as described in Example 2 at least one recombinant nucleic acid sequence encoding for a protein having a beta-1,4-galactosyltransferase activity and being able to transfer galactose on a free glucose monosaccharide to intracellularly generate lactose as e.g., described in WO2015150328. As such the sucrose is taken up or internalized into the host cell via a sucrose permease. Within the bacterial host cell, sucrose is degraded by invertase to fructose and glucose. The fructose is phosphorylated by fructokinase (e.g., frk from Zymomonas mobilis) to fructose-6-phosphate, which can then be further converted to UDP-galactose by the endogenous E. coli enzymes phosphohexose isomerase (pgi), phosphoglucomutase (pgm), glucose-1-phosphate uridylyltransferase (galU) and UDP-galactose-4-epimerase (galE). A beta-1,4-galactosyltransferase (e.g., IgtB from Neisseria meningitidis) then catalyzes the reaction UDP-galactose+glucose=>UDP+lactose. Preferably, the strain is further modified to not express the E. coli lacZ enzyme, a beta-galactosidase, which would otherwise degrade lactose. Such a strain can be used in a bio-fermentation process to produce lactose in which the strain is grown on sucrose as the sole carbon source.

### Example 10: Production of Oligosaccharides in an GM E. coli Strain

The GM E. coli strains as created in Example 8 can be further modified to produce oligosaccharides such as 3′sialyllactose, 6′sialyllactose, 2-fucosyllactose, 3′fucosyllactose, difucosyllactose, LNT or LNnT. To produce 3′ or 6′sialyllactose the GM strain need to obtain the CMP-sialic synthesis route as explained in Example 8 together with a knock-out of the lacZ gene and a knock-in of either a 3′ or a 6′ sialyltransferase enzyme. To produce 2-fucosyllactose, 3′fucosyllactose or difucosyllactose the GM strains as created in Example 8 having enhanced GDP-fucose synthesis need to obtain an alpha-1,2-fucosyltransferase and/or an alpha-1,3/1,4-fucosyltransferase together with a knock-out of the lacZ gene. To produce LNT or LNnT the GM strains as created in Example 8 having enhanced UDP-Gal and UDP-GlcNAc synthesis need to obtain a knock-out for the lacZ gene and knock-ins for a galactoside beta-1,3-N-acetylglucosaminyltransferase (lgtA) e.g., from Neisseria meningitidis and either an N-acetylglucosamide beta-1,3-galactosyltransferase (wbgO) from Escherichia coli 055:H7 for LNT production or an N-acetylglucosamide beta-1,4-galactosyltransferase (lgtB) from Neisseria meningitidis for LNnT production. Such strains can be used in bio-fermentation processes to produce the oligosaccharides and will be better in any of the following parameters: product titer, substrate uptake/conversion rate Qs (g substrate/g Biomass/h), product purity, growth speed, antifoam addition viscosity, fermentation time than sirnilar modified but non-GM E. coli strains,

### Example 11: Production of Glycolipids in an GM E. coli Strain

The GM E. coli strains as created in Example 2 and Example 3 can be used for the production of glycolipids. An example of such a glycolipid is e.g., a rhamnolipid containing one or two rhamnose residues (mono- or dirhamnolipid). The production of monorhamnolipids can be catalyzed by the enzymatic complex rhamnosyltransferase 1 (Rt1), encoded by the rhlAB operon of Pseudomonas aeruginosa, using dTDP-L-rhamnose and beta-hydroxydecanoic acid precursors. Overexpression in an GM E. coli strain of this rhlAB operon, as well as overexpression of the Pseudomonas aeruginosa rmlBDAC operon genes to increase dTDP-L-rhamnose availability, allows for monorhamnolipids production, mainly containing a C10-C10 fatty acid dimer moiety. This can be achieved in various media such as rich LB medium or minimal medium with glucose as carbon source.

1. A viable Gram-negative bacterial host cell characterized in that said host cell comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG) and Glucosylglycerol, glycan, and trehalose.

2. The host cell of claim 1, wherein the reduced or abolished synthesis is provided by a mutation in any one or more glycosyltransferase(s) involved in the synthesis of any one of the poly-N-acetyl-glucosamine (PNAG), Enterobacterial Common Antigen (ECA), cellulose, colanic acid, core oligosaccharides, Osmoregulated Periplasmic Glucans and Glucosylglycerol (OPG), glycan, and trehalose.

3. The host cell of any one of claim 1, wherein the reduced or abolished synthesis is provided by a mutation in the expression or the coding sequence of all non-essential or predicted non-essential glycosyltransferases of the host cell.

4. The host cell according to claim 3, wherein the mutation in the expression or the coding sequence provides for a deletion or lower expression of the glycosyltransferases.

5. The host cell according to claim 1, wherein the host cell is devoid of all non-essential glycosyltransferases.

6. The host cell of claim 1, wherein
the PNAG synthesis is reduced or abolished by mutation in the expression or the coding sequence of any one or more of the glycosyltransferase genes encoding poly-N-acetyl-D-glucosamine synthase subunits, or the PNAG synthesis is reduced or abolished by any one or more of i) over-expression of a carbon storage regulator encoding gene, ii) deletion of a Na⁺/H⁺ antiporter regulator encoding gene or iii) deletion of the sensor histidine kinase encoding gene,
the ECA synthesis is reduced or abolished by mutation in the expression or the coding sequence of any one or more of the glycosyltransferase genes encoding UDP-N-acetylglucosamine-undecaprenyl-phosphate N-acetylglucosaminephosphotransferase, Fuc4NAc (4-acetamido-4,6-dideoxy-D-galactose) transferase or UDP-N-acetyl-D-mannosaminuronic acid transferase,
the cellulose synthesis is reduced or abolished by mutation in the expression or the coding sequence of any one or both glycosyltransferase genes encoding the cellulose synthase catalytic subunits or the cellulose biosynthesis protein,
the colanic acid synthesis is reduced or abolished by mutation in the expression or the coding sequence of any one or more of the glycosyltransferase genes encoding colanic acid biosynthesis glucuronosyltransferase, colanic acid biosynthesis galactosyltransferase, colanic acid biosynthesis fucosyltransferase, UDP-glucose:undecaprenyl-phosphate glucose-1-phosphate transferase or putative colanic biosynthesis glycosyl transferase,
the core oligosaccharides synthesis is reduced or abolished by mutation of any one or more of the glycosyltransferase genes encoding UDP-glucuronate:LPS(HepIII) glycosyltransferase, ADP-heptose-LPS heptosyltransferase 2, ADP-heptose:LPS heptosyltransferase 1, putative ADP-heptose:LPS heptosyltransferase 4, lipopolysaccharide core biosynthesis protein, UDP-glucose:(glucosyl)LPS α-1,2-glucosyltransferase, UDP-D-glucose:(glucosyl)LPS α-1,3-glucosyltransferase, UDP-D-galactose:(glucosyl)lipopolysaccharide-1,6-D-galactosyltransferase, lipopolysaccharide glucosyltransferase I, lipopolysaccharide core heptosyltransferase 3 or β-1,6-galactofuranosyltransferase, undecaprenyl-phosphate 4-deoxy-4-formamido-L-arabinose transferase, lipid IVA 4-amino-4-deoxy-L-arabinosyltransferase, bactoprenol glucosyl transferase, putative glycosyltransferases or putative family 2 glycosyltransferase,
the OPG and Glucosylglycerol synthesis is reduced or abolished by mutation of any one or more of the glycosyltransferase genes encoding the osmoregulated periplasmic glucans (OPG) biosynthesis protein G, OPG biosynthesis protein H or glucosylglycerate phosphorylase,
the glycan synthesis is reduced or abolished by mutation of any one or more of the glycosyltransferase genes encoding glycogen synthase, 1,4-α-glucan branching enzyme or 4-α-glucanotransferase, and
the trehalose synthesis is reduced or abolished by mutation of the glycosyltransferase gene encoding trehalose-6-phosphate synthase.

7. The host cell of claim 1, wherein the PNAG synthesis is reduced or abolished by mutation of the genes pgaC or pgaD, or the PNAG synthesis is reduced or abolished by any one or more of i) over-expression of the csrA encoding gene, ii) deletion of the regulator encoding gene NhaR or iii) deletion of the kinase encoding gene resC, the ECA synthesis is reduced or abolished by mutation of any one or more of the genes rfe, rffT or rffM, the cellulose synthesis is reduced or abolished by mutation of the genes bcsA, bcsB or bcsC, the colanic acid synthesis is reduced or abolished by mutation of any one or more of the genes wcaA, wcaC, wcaE, wcaI, wcaJ or wcaL, the core oligosaccharides synthesis is reduced or abolished by mutation of any one or more of the genes waaH, waaF, waaC, waaU, waaZ, waaJ, waaO, waaB, waaS, waaG, waaQ, wbbI, arnC, arnT, yaiP, yfdH or wbbK, the OPG and Glucosylglycerol synthesis is reduced or abolished by mutation of the genes opgG, opgH or ycjM, the glycan synthesis is reduced or abolished by mutation of any one or more of the genes glgA, glgB or malQ, the trehalose synthesis is reduced or abolished by mutation of the otsA gene.

8. Host cell according to claim 1, wherein the host cell is selected from the group consisting of Escherichia spp., Shigella spp., Salmonella spp., Campylobacter spp., Neisseria spp., Moraxella spp., Stenotrophomonas spp., Bdellovibrio spp., Acinetobacter spp., Enterobacter spp., Haemophilus spp., Aeromonas spp., Francisella spp., Yersinia spp., Klebsiella spp., Bordetella spp., Legionella spp., Citrobacter spp., Chlamydia spp., Brucella spp., Pseudomonas spp., Helicobacter spp. and Vibrio spp.

9. (canceled)

10. Host cell according to claim 8, wherein the host cell is E. coli.

11. Host cell according to claim 1, wherein the host cell is selected from the group consisting of K-12 strain, W3110, MG1655, B/r, BL21, O157:h7, 042, 101-1,1180, 1357, 1412, 1520, 1827-70, 2362-75, 3431, 53638, 83972, 929-78, 98NK2, ABU 83972, B, B088, B171, B185, B354, B646, B7A, C, c7122, CFT073, DH1, DH5a, E110019, E128010, E74/68, E851/71, EAEC 042, EPECa11, EPECa12, EPECa14, ETEC, H10407, F11, F18+, FVEC1302, FVEC1412, GEMS_EPEC1, HB101, HT115, KO11, LF82, LT-41, LT-62, LT-68, MS107-1, MS119-7, MS124-1, MS 145-7, MS 79-2, MS 85-1, NCTC 86, Nissle 1917, NT:H19, NT:H40, NU14, O103:H2, O103:HNM, O103:K+, O104:H12, O108:H25, O109:H9, O111H-, O111:H19, O111:H2, O111:H21, O111:NM, O115:H-, O115:HMN, O115:K+, O119:H6, O119:UT, O124:H40, O127a:H6, O127:H6, O128:H2, O131:H25, O136:H-, O139:H28 (strain E24377A/ETEC), O13:H11, O142:H6, O145:H-, O153:H21, O153:H7, O154:H9, O157:12, O157:H-, O157:H12, O157:H43, O157:H45, O157:H7 EDL933, O157:NM, O15:NM, O177:H11, O17:K52:H18 (strain UMN026/ExPEC), O180:H-, O1:K1/APEC, O26, O26:H-, O26:H11, O26:H1L:K60, O26:NM, O41:H-, O45:K1 (strain S88/ExPEC), O51:H-, O55:H51, O55:H6, O55:H7, O5:H-, O6, O63:H6, O63:HNM, O6:K15:H31 (strain 536/UPEC), O7:K1 (strain IAI39/ExPEC), O8 (strain IAI1), O81 (strain ED1a), O84:H-, O86a:H34, O86a:H40, O90:H8, O91:H21, O9:H4 (strain HS), O9:H51, ONT:H-ONT:H25, OP50, Orough:H12, Orough:H19, Orough:H34, Orough:H37, Orough:H9, OUT:H12, OUT:H45, OUT:H6, OUT:H7, OUT:HNM, OUT:NM, RN587/1, RS218, 55989/EAEC, B/BL21, B/BL21-DE3, SE11, SMS-3-5/SECEC, UTI89/UPEC, TA004, TA155, TX1999, and Vir68.

12. Host cell according to any one of claim 1, wherein the host cell is further transformed with one or more genes of interest operably linked to a promoter and/or UTR.

13. Host cell according to claim 1, wherein the host cell is genetically modified to produce at least one bioproduct.

14. Host cell according to claim 12, wherein the gene of interest is on a plasmid or chromosome and is expressed in the host cell.

15. The host cell according to am claim 1, wherein the host cell is isolated.

16. A method of producing a bioproduct using a host cell, the method comprising the steps of:
providing a host cell, which has been genetically modified, such, that at least the host cell is able to produce the bioproduct wherein the unmodified host cell is not able to produce the bioproduct, due to the introduction of at least one heterologous gene, encoding the bioproduct or an intermediate thereof, which is expressed in the host cell; and
cultivating and/or growing the host cell in a cultivation medium enabling to production of the bioproduct thereby producing the bioproduct obtainable from the medium the host cell is cultivated in;
wherein the host cell is the host cell according to claim 1.

17. (canceled)

18. The method according to claim 16, wherein the bioproduct is a glycosylated product.

19. The method according to claim 18, wherein the bioproduct is selected from the group consisting of an oligosaccharide, a mammalian milk oligosaccharide, 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated milk oligosaccharides, neutral milk oligosaccharide sialylated milk oligosaccharides, FLNT III, and a combination of any thereof.

20. Method according to claim 18, wherein the glycosylated product is a glycolipid, a glycoprotein or oligosaccharide.

21. Method according to claim 20, wherein the bioproduct is an oligosaccharide, preferably a mammalian milk oligosaccharide, 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated milk oligosaccharides, neutral milk oligosaccharide, and/or sialylated milk oligosaccharides, FLNT III.
